(19) United States
(12) Patent Application Publication
(10) Pub. No.: US 2021/0236088 A1
(43) Pub. Date: Aug. 5, 2021

(54) GUIDE TUBE, ULTRASOUND PROBE, ULTRASOUND IMAGING SYSTEM, AND METHOD OF USING ULTRASOUND IMAGING SYSTEM

(71) Applicant: OLYMPUS CORPORATION, Tokyo (JP)

(72) Inventor: Yasuhiro IIJIMA, Tokyo (JP)

(73) Assignee: OLYMPUS CORPORATION, Tokyo (JP)

(21) Appl. No.: 17/227,554

(22) Filed: Apr. 12, 2021

Related U.S. Application Data
(63) Continuation of application No. PCT/JP2018/038558, filed on Oct. 16, 2018.

(51) Int. Cl. A61B 8/12; A61B 8/00

(52) U.S. Cl. CPC A61B 8/12; A61B 8/4483; A61B 8/4444

(57) ABSTRACT

A guide tube includes: a tube that includes a first opening arranged at a distal end; a first lumen configured to communicate with the first opening; a second opening that is arranged on an outer circumference on a proximal end side relative to the first opening; and a second lumen configured to communicate with the second opening; and a cover that is arranged on a distal end side relative to the first opening and that is arranged in a first direction when viewed from the first opening, the first direction being a direction that goes from the first lumen to the second lumen in a cross section perpendicular to a central axis of the tube.

### CROSS-REFERENCE TO RELATED APPLICATION

This application is a continuation of International Application No. PCT/JP2018/038558, filed on Oct. 16, 2018, the entire contents of which are incorporated herein by reference.

### BACKGROUND

### 1. Technical Field

The present disclosure relates to a guide tube, an ultrasound probe, an ultrasound imaging system, and a method of using the ultrasound imaging system.

### 2. Related Art

In the related art, an ultrasound imaging system (ultrasound guided puncture system) for operating a puncture needle while checking an ultrasound image of an observed region inside a subject and puncturing the observed region has been known (for example, see Japanese Laid-open Patent Publication No. 2001-104315).

The ultrasound imaging system described in Japanese Laid-open Patent Publication No. 2001-104315 includes an ultrasound probe that is provided with, in a distal end portion thereof, an ultrasound transducer for transmitting and receiving an ultrasound wave, a puncture needle for performing puncture, and a guide tube that is inserted into a subject through a treatment tool channel of an endoscope.

The guide tube includes a first opening arranged at a distal end thereof, a first lumen in which the ultrasound probe is inserted, a second opening arranged at the distal end thereof, and a second lumen in which a puncture needle is inserted.

FIG. 14 is a diagram for explaining a problem of an ultrasound imaging system 800 in the related art.

In FIG. 14, a symbol “810” denotes the above-mentioned ultrasound probe. A symbol “811” denotes a distal end portion of the ultrasound probe 810. A symbol “820” denotes the above-mentioned guide tube. A symbol “L1” denotes the above-mentioned first lumen. A symbol “L1a” denotes the above-mentioned first opening. A symbol “L2” denotes the above-mentioned second lumen. A symbol “L2a” denotes the above-mentioned second opening. In FIG. 4, examples of an observed region inside a subject include a hollow organ, such as a bronchus. A symbol “Wa” illustrated denotes a luminal wall of the hollow organ, such as the bronchus.

In the ultrasound probe 810, the guide tube 820 is inserted into the subject through a treatment tool channel (not illustrated) of an endoscope. When the guide tube 820 is inserted into the first lumen L1, a state where the distal end portion 811 protrudes from the first opening L1a is produced. In this state, an ultrasound transducer (not illustrated) that is arranged at the distal end portion 811 transmits and receives an ultrasound wave to obtain an ultrasound image of the observed region inside the subject.

Here, an outer surface of the guide tube 820 is almost in contact with the luminal wall Wa. Meanwhile, since the distal end portion 811 has an outer diameter dimension that is smaller than an outer diameter dimension of the guide tube 820, a gap Di is formed between the distal end portion 811 and the luminal wall Wa. In a state where the gap Di (air gap) exists between the distal end portion 811 and the luminal wall Wa, it is not possible to propagate an ultrasound wave between the observed region inside the subject and the ultrasound transducer in a good manner. Thus, it is not possible to obtain a desired ultrasound image. Accordingly, a technology that enables good propagation of the ultrasound wave between the observed region inside the subject and the ultrasound transducer is desired.

### SUMMARY

In some embodiments, a guide tube includes: a tube that includes a first opening arranged at a distal end; a first lumen configured to communicate with the first opening; a second opening that is arranged on an outer circumference on a proximal end side relative to the first opening; and a second lumen configured to communicate with the second opening; and a cover that is arranged on a distal end side relative to the first opening and that is arranged in a first direction when viewed from the first opening, the first direction being a direction that goes from the first lumen to the second lumen in a cross section perpendicular to a central axis of the tube.

In some embodiments, provided is an ultrasound probe that is assembled with a guide tube including a tube that includes a first opening arranged at a distal end; a first lumen configured to communicate with the first opening; a second opening that is arranged on an outer circumference on a proximal end side relative to the first opening; and a second lumen configured to communicate with the second opening; and a cover that is arranged on a distal end side relative to the first opening and that is arranged in a first direction when viewed from the first opening, the first direction being a direction that goes from the first lumen to the second lumen in a cross section perpendicular to a central axis of the tube. The ultrasound probe includes: a probe main body that includes an ultrasound transducer transmitting and receiving an ultrasound wave at a distal end portion of the probe main body, and that is to be inserted into the first lumen; and a positioning mechanism that allows the distal end portion protruding from the first opening to determine a position of the probe main body with respect to the cover in the central axis direction of the tube.

In some embodiments, an ultrasound imaging system includes: an ultrasound probe that includes an ultrasound transducer transmitting and receiving an ultrasound wave at a distal end portion of the ultrasound probe; a treatment tool configured to perform treatment on living tissue; and a guide tube that includes a tube that includes a first opening arranged at a distal end; a first lumen in which the ultrasound probe is inserted, the first lumen being configured to communicate with the first opening; a second opening that is arranged on an outer circumference on a proximal end side relative to the first opening; and a second lumen in which the treatment tool is inserted, the second lumen being configured to communicate with the second opening; and a cover that is arranged on a distal end side relative to the first opening and that is arranged in a first direction when viewed from the first opening, the first direction being a direction that goes from the first lumen to the second lumen in a cross section perpendicular to a central axis of the tube.

In some embodiments, provided is a method of using an ultrasound imaging system. The method includes: injecting an ultrasound medium that propagates an ultrasound wave to an inside of the cover of the guide tube; inserting the ultrasound probe into the first lumen; causing the distal end portion to protrude from the first opening; determining a position of the ultrasound probe in a central axis direction of the tube; and inserting the treatment tool into the second lumen.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an ultrasound imaging system according to a first embodiment;

FIG. 2 is a diagram illustrating an ultrasound probe;

FIG. 3 is a diagram illustrating a distal end portion of a guide tube;

FIG. 4 is a flowchart illustrating a method of using the ultrasound imaging system;

FIG. 5 is a diagram for explaining the method of using the ultrasound imaging system;

FIG. 6 is a diagram illustrating a cover according to a second embodiment;

FIG. 7 is a diagram illustrating a cover according to a third embodiment;

FIG. 8 is a diagram illustrating a cover according to a fourth embodiment;

FIG. 9 is a flowchart illustrating a method of using an ultrasound imaging system;

FIG. 10 is a diagram illustrating a cover according to a fifth embodiment;

FIG. 11 is a diagram illustrating a cover according to a sixth embodiment;

FIG. 12 is a diagram illustrating a cover according to a seventh embodiment;

FIG. 13 is a diagram illustrating a modification of the first to the seventh embodiments; and

FIG. 14 is a diagram for explaining a problem in the related art.

### DETAILED DESCRIPTION

Modes for carrying out the present disclosure (hereinafter, embodiments) will be described below with reference to the drawings. The present disclosure is not limited by the embodiments described below. Furthermore, in the description of the drawings, the same components are denoted by the same reference symbols.

### First Embodiment

Overall Configuration of Ultrasound Imaging System

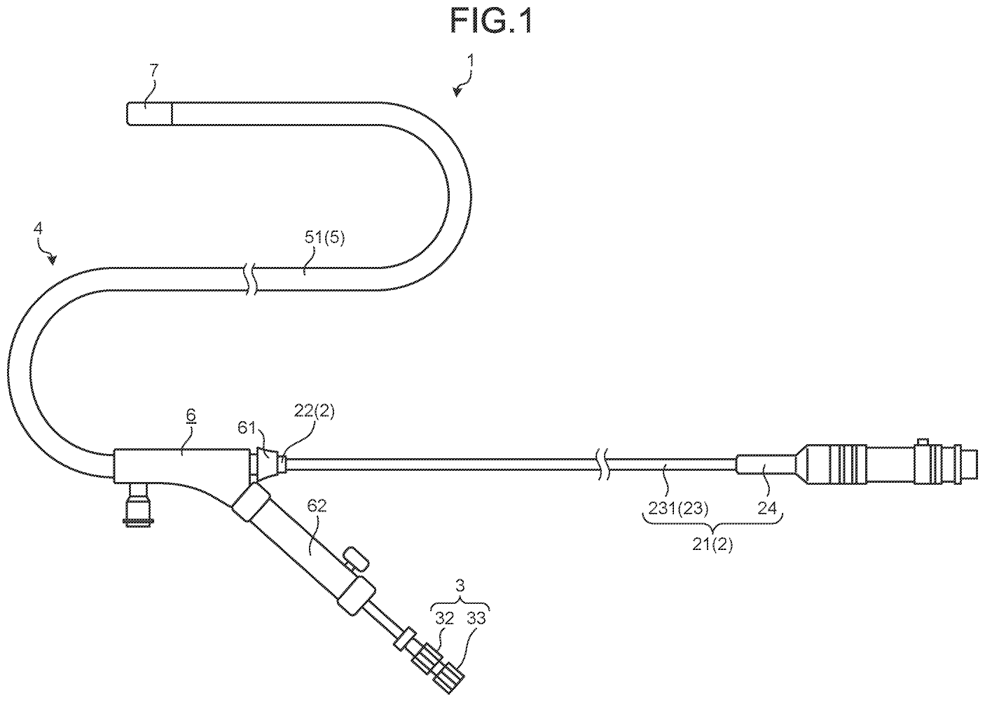

FIG. 1 is a diagram illustrating an ultrasound imaging system 1 according to a first embodiment.

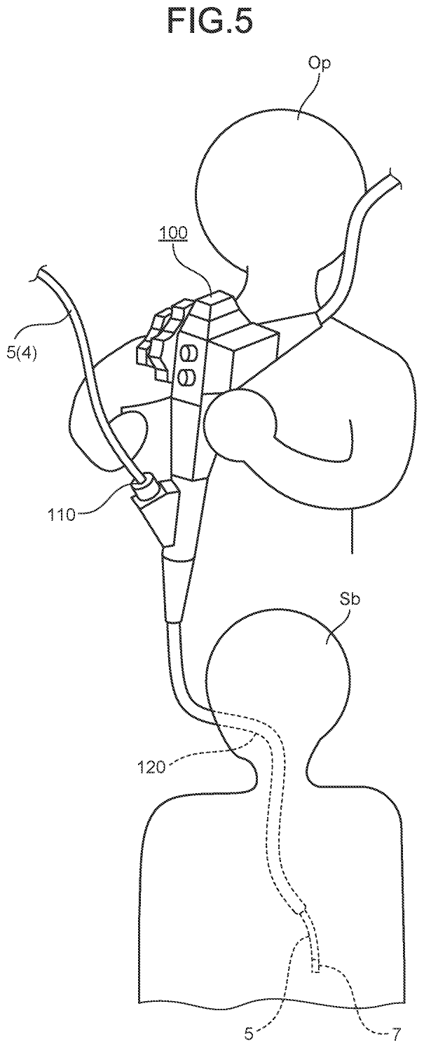

The ultrasound imaging system 1 is an ultrasound guided puncture system for operating a puncture needle while checking an ultrasound image of an observed region inside a subject Sb (see FIG. 5) and puncturing the observed region. Here, examples of the observed region inside the subject Sb include hollow organs, such as a bronchus, a pancreatic duct, and a bile duct. As illustrated in FIG. 1, the ultrasound imaging system 1 includes an ultrasound probe 2, a puncture needle 3, and a guide tube 4.

Meanwhile, a “distal end” described below indicates a distal end in an insertion direction into the subject Sb. Further, a “proximal end” described below indicates one end opposite to the distal end (one end close at hand).

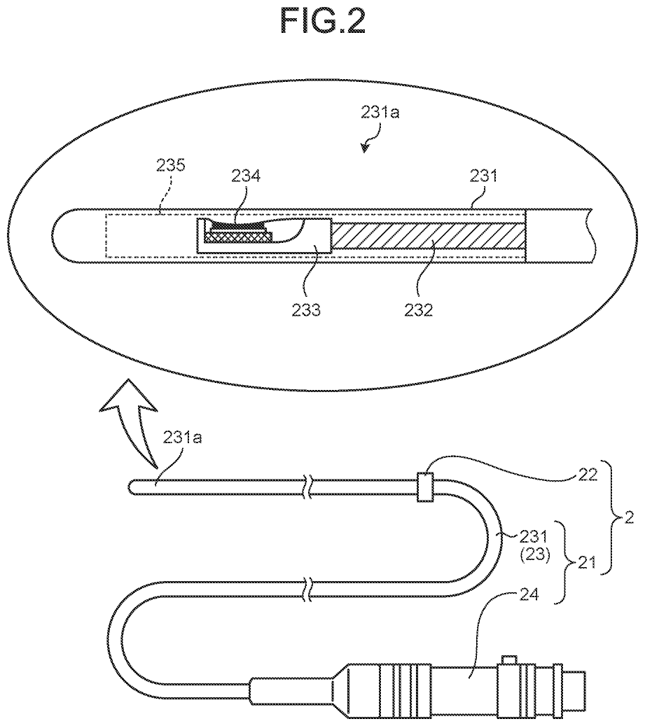

FIG. 2 is a diagram illustrating the ultrasound probe 2.

The ultrasound probe 2 is used to acquire an ultrasound image of the observed region inside the subject Sb. As illustrated in FIG. 2, the ultrasound probe 2 includes a probe main body 21 and a positioning mechanism 22.

As illustrated in FIG. 2, the probe main body 21 includes a probe insertion portion 23 and a connection portion 24.

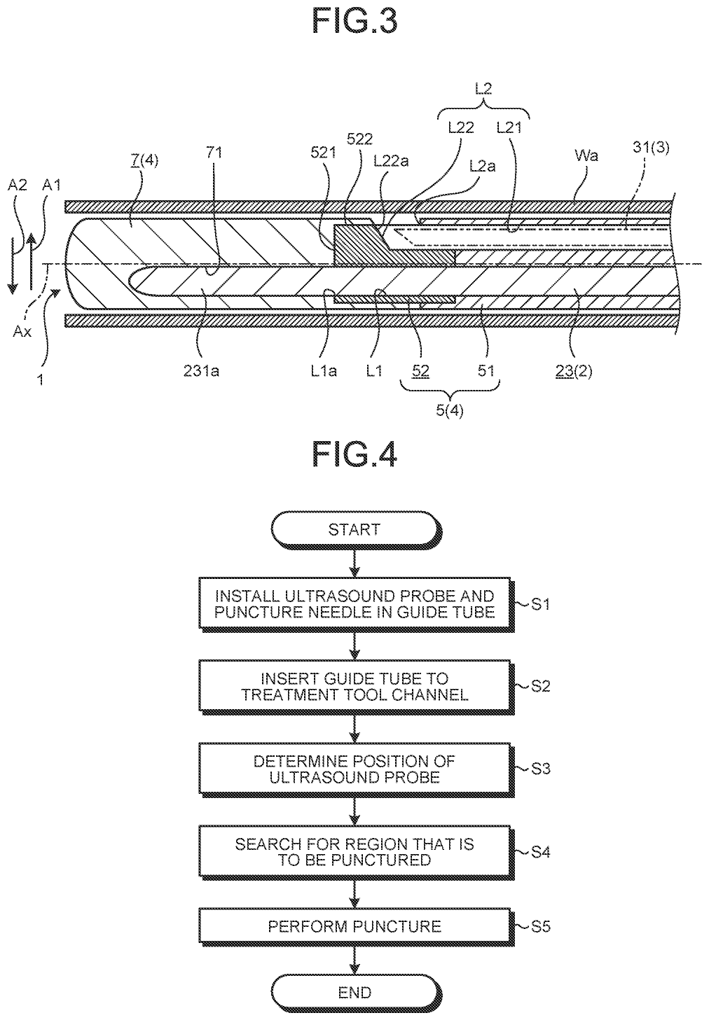

The probe insertion portion 23 is a portion to be inserted into a first lumen L1 (see FIG. 3) of the guide tube 4. The probe insertion portion 23 includes a sheath 231, a shaft 232, a housing 233, and an ultrasound transducer 234.

The sheath 231 is a flexible long and elongated member and constitutes an outer surface of the probe insertion portion 23. Examples of a material of the sheath 231 include materials, such as polyethylene, polymethylpentene, polyurethane, and polyether block amide, which can transmit an ultrasound wave.

The shaft 232 is formed in a coil shape extending from a proximal end of the sheath 231 to a distal end portion 231a (FIG. 2) of the sheath 231 inside the sheath 231. Further, the shaft 232 is configured so as to be able to rotate about a central axis of the sheath 231.

The housing 233 supports the ultrasound transducer 234 and is mounted on a distal end of the shaft 232 inside the sheath 231.

The ultrasound transducer 234 is a portion that transmits and receives an ultrasound wave. More specifically, the ultrasound transducer 234 converts a pulse signal that is output from an external ultrasound imaging device (not illustrated) to an ultrasound pulse, and transmits the ultrasound pulse to a direction away from the central axis of the sheath 231. Further, the ultrasound transducer 234 converts an ultrasound echo that is reflected by the observed region inside the subject Sb to an electrical echo signal, and outputs the echo signal to the ultrasound imaging device as described above. Furthermore, the ultrasound imaging device performs a predetermined process on the echo signal and generates an ultrasound image of the observed region inside the subject Sb. Moreover, the ultrasound image is displayed on an external display device (not illustrated).

Furthermore, the sheath 231 is filled with an ultrasound medium 235 (FIG. 2) that propagates an ultrasound wave. Examples of a material of the ultrasound medium 235 include materials, such as water, ultrasound jelly, and liquid paraffin, in which ultrasound attenuation is relatively low.

As described above, in the first embodiment, the ultrasound probe 2 is configured with an ultrasound probe of a mechanical radial type.

The connection portion 24 is a portion that is connected to a cable (not illustrated) connected to the ultrasound imaging device as described above, and is arranged on a proximal end of the probe insertion portion 23.

The positioning mechanism 22 is a portion that is mounted on an outer surface of the sheath 231, and determines a position of the probe main body 21 with respect to the guide tube 4. Meanwhile, a method of determining the position of the probe main body 21 with respect to the guide tube 4 using the positioning mechanism 22 will be described in “method of using ultrasound imaging system” to be described later.

The puncture needle 3 corresponds to a treatment tool according to the present disclosure. The puncture needle 3 is used to puncture the observed region inside the subject Sb. Further, the puncture needle 3 includes a puncture needle main body 31 (see FIG. 3) that is inserted into a second lumen L2 (see FIG. 3) of the guide tube 4, and further includes a puncture needle lure cap 32 (FIG. 1) and a lure cap cover 33 (FIG. 1), each of which is mounted on a proximal end of the puncture needle main body 31.

The guide tube 4 is inserted into the subject Sb through a treatment tool channel (not illustrated) of an endoscope 100 (see FIG. 5), and guides the probe insertion portion 23 and the puncture needle main body 31 to the inside of the subject Sb. Here, the treatment tool channel is a hole that penetrates from a treatment tool insertion opening 110 (see FIG. 5) to a distal end of an endoscope insertion portion 120 (see FIG. 5) of the endoscope 100, and that allows the guide tube 4 or the like to be inserted from the treatment tool insertion opening 110.

A detailed configuration of the guide tube 4 will be described below.

Configuration of Guide Tube

FIG. 3 is a cross-sectional view of a distal end portion of the guide tube 4. Specifically, FIG. 3 is a cross-sectional view of the guide tube 4 taken along a flat surface that goes along a central axis Ax of a tube main body 51. Meanwhile, a symbol “Wa” illustrated in FIG. 3 denotes a luminal wall of a hollow organ, such as a bronchus.

As illustrated in FIG. 1 or FIG. 3, the guide tube 4 includes a tube 5, a guide tube connector 6 (FIG. 1), and a cover 7.

The tube 5 is a portion that is inserted into the subject Sb through the treatment tool channel of the endoscope 100, and, as illustrated in FIG. 3, includes the tube main body 51 and a rigid member 52.

The tube main body 51 is a flexible long and elongated columnar member.

The rigid member 52 is a hard member made with a resin material, and, as illustrated in FIG. 3, mounted on a distal end of the tube main body 51. The rigid member 52 is a columnar member coaxial with the central axis Ax (FIG. 3) of the tube main body 51, and has an outer diameter dimension that is smaller than an outer diameter dimension of the tube main body 51.

Further, as illustrated in FIG. 3, the first lumen L1 in which the probe insertion portion 23 is inserted and the second lumen L2 in which the puncture needle main body 31 is inserted are arranged inside the tube 5.

The first lumen L1 is a hole that penetrates from a proximal end of the tube main body 51 to a distal end 521 (FIG. 3) of the rigid member 52. In other words, as illustrated in FIG. 3, the first lumen L1 has a first opening L1a at the distal end 521 of the rigid member 52. More specifically, the first lumen L1 has a circular cross section that is linearly extended from the proximal end of the tube main body 51 to the distal end 521 of the rigid member 52 along the central axis Ax, and has an inner diameter dimension that is slightly larger than an outer diameter dimension of the probe insertion portion 23 (the sheath 231).

The second lumen L2 is a hole that penetrates from the proximal end of the tube main body 51 to an outer periphery 522 (see FIG. 3) of the rigid member 52 around the central axis Ax. In other words, as illustrated in FIG. 3, the second lumen L2 has a second opening L2a in the outer periphery 522 of the rigid member 52. More specifically, the second lumen L2 includes a lumen main body L21, which has a circular cross section that is linearly extended from the proximal end of the tube main body 51 to the inside of the rigid member 52 along the central axis Ax, and an inclined portion L22, which communicates with the lumen main body L21, which has a circular cross section that is extended toward an upper left side across the central axis Ax in FIG. 3, and which is connected to the second opening L2a. In other words, the inclined portion L22 has an inclined surface L22a that is extended in the upper left direction toward the second opening L2a in FIG. 3.

As illustrated in FIG. 1, the guide tube connector 6 is arranged on the proximal end of the tube main body 51. A proximal end side of the guide tube connector 6 is divided into two portions, i.e., a probe insertion portion 61 and a puncture handle 62.

Further, while detailed illustration in the drawings is omitted, a probe insertion lumen and a puncture needle insertion lumen are arranged inside the guide tube connector 6.

The probe insertion lumen penetrates from a proximal end of the probe insertion portion 61 to a distal end of the guide tube connector 6, and communicates with the first lumen L1.

The puncture needle insertion lumen penetrates from a proximal end of the puncture handle 62 to the distal end of the guide tube connector 6, and communicates with the second lumen L2.

As illustrated in FIG. 3, the cover 7 is mounted on the distal end 521 of the tube 5 (the rigid member 52). In the first embodiment, the cover 7 is a columnar member formed of a solid material coaxial with the central axis Ax, and has an outer diameter dimension that is equal to the outer diameter dimension of the tube main body 51. Examples of a material of the cover 7 include, similarly to the sheath 231, materials, such as polyethylene, polymethylpentene, polyurethane, and polyether block amide, which can transmit an ultrasound wave.

In the cover 7, as illustrated in FIG. 3, an insertion hole 71 is arranged at a position facing the first opening L1a. The insertion hole 71 has a circular cross section that is linearly extended from a proximal end to a distal end of the cover 7, and has an inner diameter dimension that is equal to the inner diameter dimension of the first lumen L1. Meanwhile, in a state in which the cover 7 is mounted on the distal end 521 of the tube 5, the first opening L1a is covered with the cover 7. In contrast, the second opening L2a is located on the proximal end side relative to the cover 7 and is not covered with the cover 7.

Here, an arrow A1 illustrated in FIG. 3 corresponds to a first direction according to the present disclosure, which is a direction that goes from the first lumen L1 to the second lumen L2 in a cross section perpendicular to the central axis Ax (see FIG. 13). Further, an arrow A2 illustrated in FIG. 3 corresponds to a second direction according to the present disclosure, which is a direction opposite to the first direction A1. Furthermore, as illustrated in FIG. 3, a part of the cover 7 is arranged side by side with a distal end portion 231a, which protrudes from the first opening L1a, in the first direction A1. In the first embodiment, the cover 7 is extended along the entire circumference of the distal end portion 231a protruding from the first opening L1a, in a circumferential direction around the central axis of the distal end portion 231a.

Method of Using Ultrasound Imaging System

A method of using the ultrasound imaging system 1 as described above will be described below.

FIG. 4 is a flowchart illustrating the method of using the ultrasound imaging system 1. FIG. 5 is a diagram for explaining the method of using the ultrasound imaging system 1.

Meanwhile, in the following, it is assumed that the endoscope insertion portion 120 is inserted into the subject Sb in advance, and only operation (use method) of the ultrasound imaging system 1 will be described.

First, a worker, such as an operator Op, installs the ultrasound probe 2 and the puncture needle 3 in the guide tube 4 (Step S1).

Specifically, at Step S1, the worker, such as the operator Op, inserts the probe insertion portion 23 of the ultrasound probe 2 in the first lumen L1 from the proximal end of the probe insertion portion 61 of the guide tube 4 via the probe insertion lumen. Further, the worker, such as the operator Op, inserts the puncture needle main body 31 of the puncture needle 3 to the second lumen L2 from the proximal end of the puncture handle 62 of the guide tube 4 via the puncture needle insertion lumen.

Meanwhile, at Step S1, it may be possible to first wet the distal end portion 231a of the probe insertion portion 23 with an ultrasound medium and thereafter insert the probe insertion portion 23 into the guide tube 4 (the first lumen L1).

After Step S1, as illustrated in FIG. 5, the worker, such as the operator Op, inserts the tube 5 of the guide tube 4 into the treatment tool channel from the treatment tool insertion opening 110 of the endoscope 100 (Step S2). Accordingly, a distal end portion of the tube 5 protrudes from the distal end of the endoscope insertion portion 120.

After Step S2, the worker, such as the operator Op, pushes the probe insertion portion 23 toward the distal end until the positioning mechanism 22 comes into contact with the proximal end of the probe insertion portion 61 (Step S3). Accordingly, a position of the distal end portion 231a of the probe insertion portion 23 is determined inside the cover 7 (the insertion hole 71). In other words, the distal end portion 231a protrudes from the first opening L1a and the position thereof is determined at a certain position side by side with the cover 7 in the second direction A2 (FIG. 3).

After Step S3, the worker, such as the operator Op, operates an external ultrasound imaging device (not illustrated), and causes the ultrasound transducer 234 to start to transmit and receive an ultrasound wave. Then, the worker, such as the operator Op, searches for a region that is to be punctured by the puncture needle 3 while checking an ultrasound image of the observed region inside the subject Sb displayed on an external display device (not illustrated) (Step S4).

After Step S4, the worker, such as the operator Op, operates the puncture handle 62, and pushes the puncture needle main body 31 toward the distal end (Step S5). Accordingly, the puncture needle main body 31 is guided along the inclined surface L22a, protrudes from the second opening L2a in the upper left direction in FIG. 3, and punctures the region searched for at Step S4.

According to the first embodiment as described above, it is possible to achieve effects as described below.

The ultrasound imaging system 1 (the guide tube 4) according to the first embodiment includes the cover 7, a part of which is arranged side by side with the distal end portion 231a of the ultrasound probe 2, which protrudes from the first opening L1a, in the first direction A1. In other words, the cover 7 is arranged side by side with the distal end portion 231a at a side of a region to be punctured by the puncture needle 3. In particular, the cover 7 has the same outer shape as that of the tube main body 51 in a cross section perpendicular to the central axis Ax. Therefore, the cover 7 is located between the distal end portion 231a and the luminal wall Wa, so that an interspace (airspace) is eliminated.

Therefore, according to the ultrasound imaging system 1 (the guide tube 4) of the first embodiment, it is possible to allow an ultrasound wave to propagate between the observed region inside the subject and the ultrasound transducer 234 in a good manner. In other words, it is possible to obtain a desired ultrasound image, so that it is possible to smoothly puncture a desired region while checking the ultrasound image.

Furthermore, the ultrasound imaging system 1 (the guide tube 4) according to the first embodiment includes the inclined surface L22a in the second lumen L2. Therefore, it is possible to guide a protruding direction of the puncture needle 3 by the inclined surface L22a, so that it is possible to puncture a desired region.

Moreover, in the ultrasound imaging system 1 (the guide tube 4) according to the first embodiment, the cover 7 is arranged on the distal end side relative to the second opening L2a. In other words, the second opening L2a is not covered with the cover 7. Therefore, if the second opening L2a is covered with the cover 7, it is necessary to perform a process on the cover 7 to allow the puncture needle 3 to protrude; however, according to the first embodiment, this process is not needed, and it is possible to construct the cover 7 with a simple structure.

Furthermore, the ultrasound probe 2 according to the first embodiment includes the positioning mechanism 22 that determines the position of the probe main body 21 at a certain position at which the distal end portion 231a protruding from the first opening L1a is arranged side by side with the cover 7 in the second direction A2. Therefore, the cover 7 is reliably located between the distal end portion 231a and the lumen wall Wa, so that it is possible to achieve the effect as described above, such as “it is possible to propagate an ultrasound wave between the observed region inside the subject and the ultrasound transducer 234 in a good manner”, in a preferable manner.

### Second Embodiment

A second embodiment will be described below.

In the description below, the same components as those of the first embodiment as described above are denoted by the same reference symbols, and explanation thereof will be omitted or simplified.

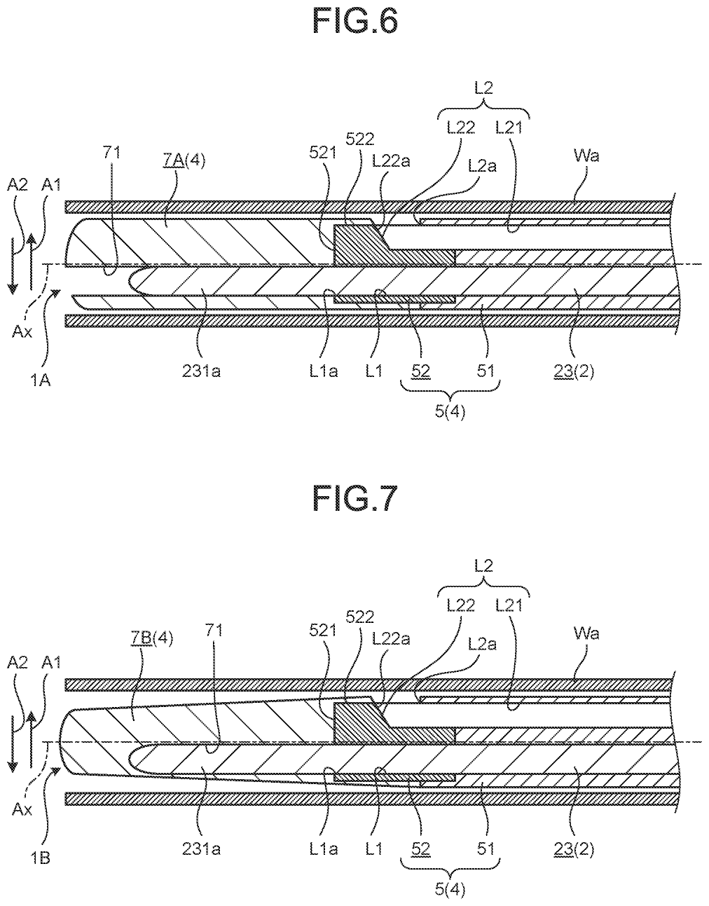

FIG. 6 is a diagram illustrating a cover 7A according to the second embodiment. Specifically, FIG. 6 is a cross-sectional view corresponding to FIG. 3.

In an ultrasound imaging system 1A according to the second embodiment, as illustrated in FIG. 6, the cover 7A that has a different shape from that of the cover 7 is adopted, instead of the cover 7, in the ultrasound imaging system 1 explained in the first embodiment described above.

The cover 7A illustrated in FIG. 6 is different from the cover 7 only in that the insertion hole 71 penetrates to a distal end of the cover 7A.

Meanwhile, a method of using the ultrasound imaging system 1A is the same as the method of using the ultrasound imaging system 1 explained in the first embodiment described above.

According to the second embodiment as described above, it is possible to achieve effects as described below, in addition to the same effects as those of the first embodiment described above.

In the cover 7A according to the second embodiment, the insertion hole 71 penetrates from a proximal end to the distal end of the cover 7A. In other words, the insertion hole 71 does not limit movement of the distal end portion 231a toward the distal end. Therefore, an observable range using the ultrasound probe 2 is extended, so that it is possible to improve usability.

### Third Embodiment

A third embodiment will be described below.

In the description below, the same components as those of the first embodiment as described above are denoted by the same reference symbols, and explanation thereof will be omitted or simplified.

FIG. 7 is a diagram illustrating a cover 7B according to the third embodiment. Specifically, FIG. 7 is a cross-sectional view corresponding to FIG. 3.

In an ultrasound imaging system 1B according to the third embodiment, as illustrated in FIG. 7, the cover 7B that has a different shape from that of the cover 7 is adopted, instead of the cover 7, in the ultrasound imaging system 1 explained in the first embodiment described above.

The cover 7B illustrated in FIG. 7 is different from the cover 7 only in that it has a circular truncated cone shape that is tapered toward a distal end.

Meanwhile, a method of using the ultrasound imaging system 1B is the same as the method of using the ultrasound imaging system 1 explained in the first embodiment described above.

According to the third embodiment as described above, it is possible to achieve effects as described below, in addition to the same effects as those of the first embodiment described above.

The cover 7B according to the third embodiment has the circular truncated cone shape that is tapered toward the distal end. Therefore, it is possible to improve insertion performance into the subject.

### Fourth Embodiment

A fourth embodiment will be described below.

In the description below, the same components as those of the first embodiment as described above are denoted by the same reference symbols, and explanation thereof will be omitted or simplified.

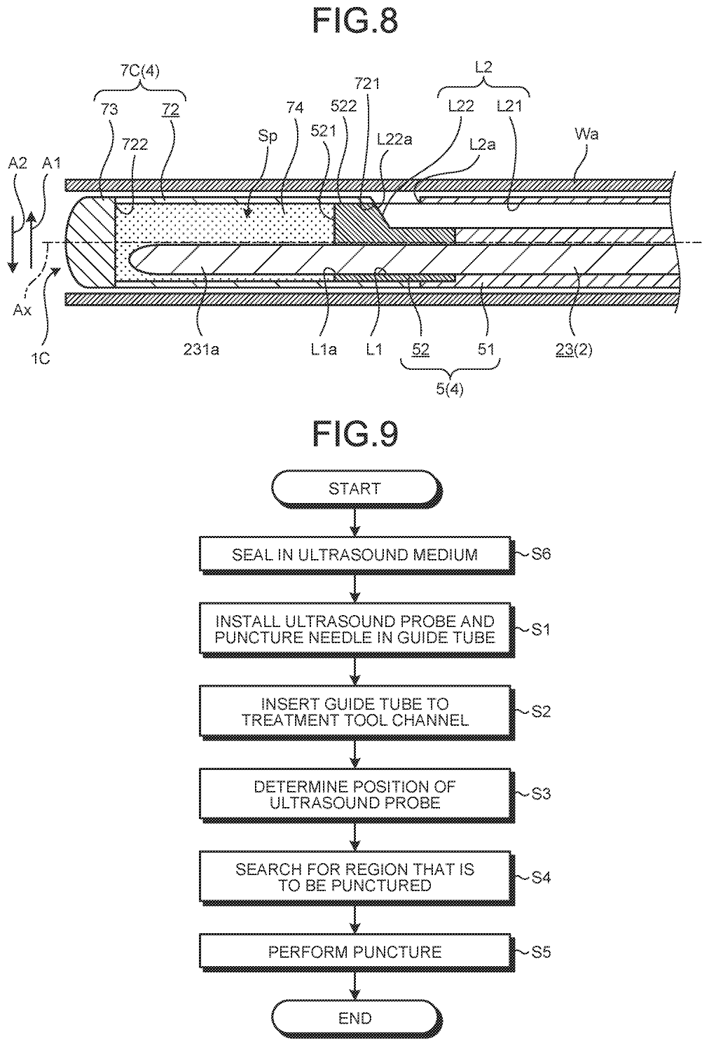

FIG. 8 is a diagram illustrating a cover 7C according to the fourth embodiment. Specifically, FIG. 8 is a cross-sectional view corresponding to FIG. 3.

In an ultrasound imaging system 1C according to the fourth embodiment, as illustrated in FIG. 8, the cover 7C that has a different shape from that of the cover 7 is adopted, instead of the cover 7, in the ultrasound imaging system 1 explained in the first embodiment described above.

Examples of a material of the cover 7C include materials, such as polyethylene, polymethylpentene, polyurethane, and polyether block amide, which can transmit an ultrasound wave. As illustrated in FIG. 8, the cover 7C includes a cover main body 72 and a cap 73.

The cover main body 72 is a cylindrical member coaxial with the central axis Ax, and has an outer diameter dimension that is equal to the outer diameter dimension of the tube main body 51. Further, in the cover main body 72, the rigid member 52 is fitted to an opening 721 on the proximal end side.

The cap 73 is detachably attachable to a distal end of the cover main body 72. Further, the cap 73 closes an opening 722 of the cover main body 72 on the distal end side in a state of being attached to the distal end of the cover main body 72. The opening 722 corresponds to a third opening according to the present disclosure.

As described above, the cover 7C is configured with a hollow member that has a space Sp in which the distal end portion 231a protruding from the first opening L1a is inserted. Meanwhile, in a state in which the cover 7C is mounted on the distal end 521 of the tube 5, the first opening L1a is covered with the cover 7C. In contrast, the second opening L2a is located on the proximal end side relative to the cover 7C, and is not covered with the cover 7C.

Meanwhile, similarly to the cover 7, a part of the cover 7C is arranged side by side with the distal end portion 231a, which protrudes from the first opening L1a, in the first direction A1.

A method of using the ultrasound imaging system 1C will be described below.

FIG. 9 is a flowchart illustrating the method of using the ultrasound imaging system 1C.

Meanwhile, as illustrated in FIG. 9, the method of using the ultrasound imaging system 1C according to the fourth embodiment is different from the method of using the ultrasound imaging system 1 explained in the first embodiment described above only in that Step S6 is added. Therefore, in the following, only Step S6 is explained.

Step S6 is performed before Step S1.

Specifically, at Step S6, the worker, such as the operator Op, removes the cap 73 from the cover main body 72, and fills the space Sp with an ultrasound medium 74 (FIG. 8) from the opening 722 on the distal end side. Here, examples of a material of the ultrasound medium 74 include, similarly to the ultrasound medium 235, materials, such as water, ultrasound jelly, and liquid paraffin, in which ultrasound attenuation is relatively low. Then, the worker, such as the operator Op, attaches the cap 73 to the cover main body 72 and keeps the ultrasound medium 74 sealed in the space Sp. Thereafter, the worker, such as the operator Op, performs Step S1.

Meanwhile, as described above, the ultrasound medium 74 may be injected to the inside of the space Sp from the opening 722 on the distal end side after removing the cap 73 from the cover main body 72, or may be injected to the inside of the space Sp from the opening 721 (the probe insertion portion 61) on the proximal end side in a state in which the cap 73 is attached to the cover main body 72.

According to the fourth embodiment as described above, it is possible to achieve effects as described below, in addition to the same effects as those of the first embodiment described above.

The cover 7C according to the fourth embodiment is configured with a hollow member including the cover main body 72 and the cap 73, and the ultrasound medium 74 is sealed in the internal space Sp. Therefore, it is possible to reduce a thickness of a side wall portion of the cover main body 72 that is located in a region in which an ultrasound wave is transmitted and received in the ultrasound transducer 234. In other words, it is possible to reduce ultrasound attenuation due to the cover 7C, so that it is possible to propagate ultrasound waves between the observed region inside the subject and the ultrasound transducer 234 in a better manner.

Furthermore, the ultrasound medium 74 can be sealed in the space Sp by the cap 73, so that it is possible to reliably retain the ultrasound medium 74 in a space (in the space Sp) between the observed region of the subject and the ultrasound transducer 234.

### Fifth Embodiment

A fifth embodiment will be described below.

In the description below, the same components as those of the fourth embodiment as described above are denoted by the same reference symbols, and explanation thereof will be omitted or simplified.

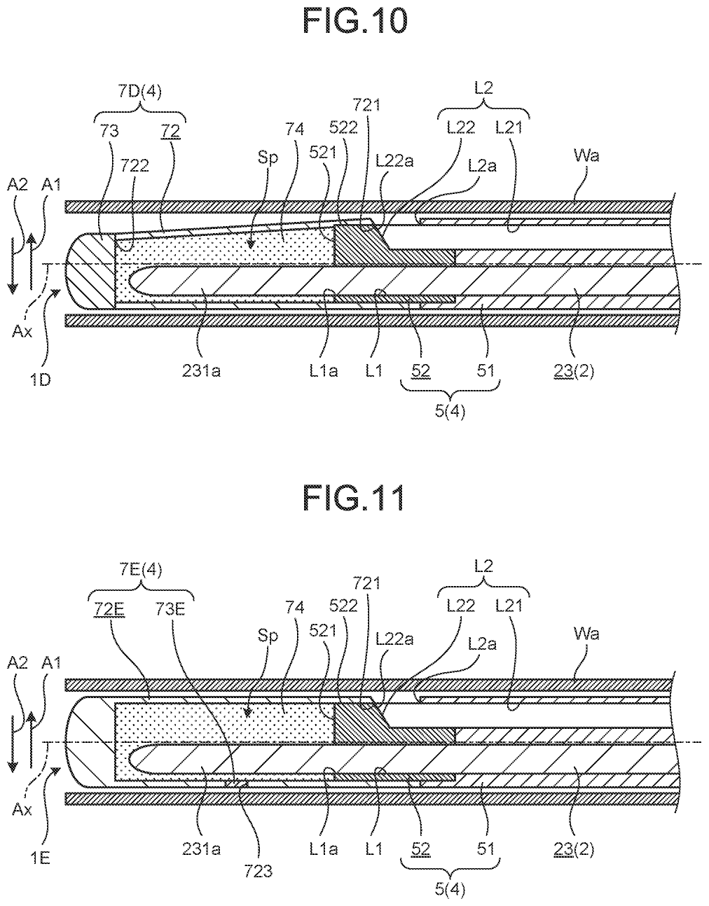

FIG. 10 is a diagram illustrating a cover 7D according to the fifth embodiment. Specifically, FIG. 10 is a cross-sectional view corresponding to FIG. 8.

In an ultrasound imaging system 1D according to the fifth embodiment, as illustrated in FIG. 10, the cover 7D that has a different shape from that of the cover 7C is adopted, instead of the cover 7C, in the ultrasound imaging system 1C explained in the fourth embodiment described above.

The cover 7D illustrated in FIG. 10 is different from the cover 7C only in that an entire shape thereof is a circular truncated cone shape that is tapered toward a distal end.

Meanwhile, a method of using the ultrasound imaging system 1D is the same as the method of using the ultrasound imaging system 1C explained in the fourth embodiment described above.

According to the fifth embodiment as described above, it is possible to achieve the same effects as those of the third and the fourth embodiments as described above.

### Sixth Embodiment

A sixth embodiment will be described below.

In the description below, the same components as those of the fourth embodiment as described above are denoted by the same reference symbols, and explanation thereof will be omitted or simplified.

FIG. 11 is a diagram illustrating a cover 7E according to the sixth embodiment. Specifically, FIG. 11 is a cross-sectional view corresponding to FIG. 8.

In an ultrasound imaging system 1E according to the sixth embodiment, as illustrated in FIG. 11, the cover 7E that has a different shape from that of the cover 7C is adopted, instead of the cover 7C, in the ultrasound imaging system 1C explained in the fourth embodiment described above.

The cover 7E is configured with the same material as that of the cover 7C. As illustrated in FIG. 11, the cover 7E includes a cover main body 72E and a cap 73E.

The cover main body 72E has a shape in which the cover main body 72 and the cap 73 explained in the fourth embodiment described above are integrated.

In the cover main body 72E, as illustrated in FIG. 11, an opening 723 that allows communication between the inside and the outside of the space Sp is formed on a side wall portion that constitutes an outer periphery around the central axis Ax. The opening 723 corresponds to the third opening according to the present disclosure. Meanwhile, it is preferable to form the opening 723 in a region other than a side wall portion in the first direction A1 where the puncture needle main body 31 protrudes from the second opening L2a among side wall portions that constitute the outer periphery around the central axis Ax in the cover main body 72E.

The cap 73E is detachably attachable to the cover main body 72E. Further, the cap 73E closes the opening 723 in a state of being attached to the cover main body 72E.

In other words, the cover 7E is different from the cover 7C explained in the fourth embodiment described above in that the positions of the third opening according to the present disclosure and the cap are changed.

Meanwhile, a method of using the ultrasound imaging system 1E is the same as the method of using the ultrasound imaging system 1C explained in the fourth embodiment described above.

According to the sixth embodiment as described above, it is possible to achieve the same effects as those of the fourth embodiment described above.

### Seventh Embodiment

A seventh embodiment will be described below.

In the description below, the same components as those of the fourth embodiment as described above are denoted by the same reference symbols, and explanation thereof will be omitted or simplified.

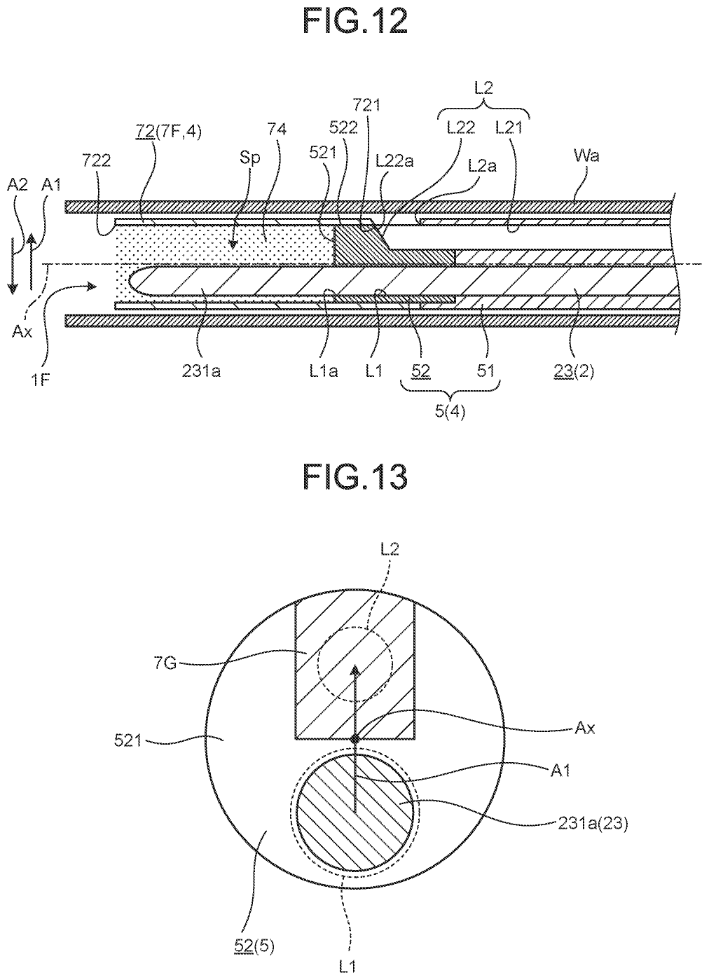

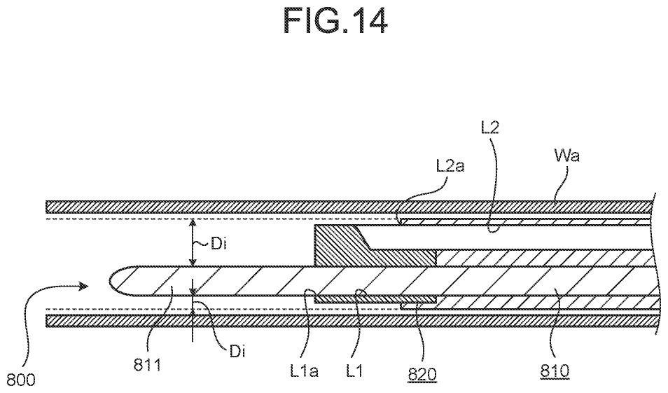

FIG. 12 is a diagram illustrating a cover 7F according to the seventh embodiment. Specifically, FIG. 12 is a cross-sectional view corresponding to FIG. 8.

In an ultrasound imaging system 1F according to the seventh embodiment, as illustrated in FIG. 12, the cover 7F that has a different shape from that of the cover 7C is adopted, instead of the cover 7C, in the ultrasound imaging system 1C explained in the fourth embodiment described above.

The cover 7F illustrated in FIG. 12 is different from the cover 7C only in that the cap 73 is omitted.

Meanwhile, a method of using the ultrasound imaging system 1F is different from the method of using the ultrasound imaging system 1C explained in the fourth embodiment described above only in that, at Step S6, the space Sp is filled with the ultrasound medium 74 but the ultrasound medium 74 is not sealed in the space Sp by the cap 73. In this case, to retain the ultrasound medium 74 in the space Sp, it is preferable to adopt a material with a relatively high viscosity (for example, ultrasound jelly).

According to the seventh embodiment as described above, it is possible to achieve the same effects as those of the second and the fourth embodiments. Meanwhile, even in the fifth and the sixth embodiments as described above, it may be possible to omit the caps 73 and 73E, similarly to the seventh embodiment.

### Other Embodiments

While the embodiments of the present disclosure have been explained above, the present disclosure is not limited to only the first to the seventh embodiments as described above.

FIG. 13 is a diagram illustrating a modification of the first to the seventh embodiments. Specifically, FIG. 13 is a cross-sectional view of the distal end portion of the guide tube 4 taken along a plane perpendicular to the central axis Ax.

In the first to the seventh embodiments as described above, a part of the cover 7 (7A to 7F) is arranged side by side with the distal end portion 231a, which protrudes from the first opening, L1a in the first direction A1; however, embodiments are not limited to this example. For example, as illustrated in FIG. 13, it may be possible to adopt a cover 7G, the entire part thereof is arranged side by side with a distal end portion 231a, which protrudes from the first opening L1a, in the first direction A1.

In the first to the seventh embodiments as described above, the ultrasound probe 2 is configured with an ultrasound probe of a mechanical radial type; however, embodiments are not limited to this example, and it may be possible to adopt an ultrasound probe of a different type.

In the first to the seventh embodiments as described above, the puncture needle 3 is adopted as a treatment tool according to the present disclosure; however, embodiments are not limited to this example, and it may be possible to adopt a different treatment tool, such as forceps, a spoon, or a brush.

In the first to the seventh embodiments as described above, the probe insertion portion 23 and the tube 5 have outer surfaces with circular cross sections; however, embodiments are not limited to this example, and it may be possible to adopt an outer surface with a different-shaped cross section. The same applies to the first and the second lumens L1 and L2.

In the ultrasound imaging system 1 (1A to 1F) according to the first to the seventh embodiments as described above, it may be possible to arrange a fixing mechanism, such as a chuck, that, after the position of the probe main body 21 is determined with respect to the guide tube 4 by the positioning mechanism 22, fixes the determined position.

In the first to the seventh embodiments as described above, the methods of using the ultrasound imaging system 1 (1A to 1F) are not limited to the flows illustrated in FIG. 4 and FIG. 9, and sequences may be modified as long as there is no contradiction.

In the fourth to the seventh embodiments as described above, it may be possible to arrange a membrane that closes the opening 721 or the first opening L1a on the proximal end side in order to prevent the ultrasound medium 74 from entering the first lumen L1 from the space Sp. In this case, the membrane is torn by the distal end portion 231a when the distal end portion 231a protrudes from the first opening L1a.

According to the guide tube, the ultrasound probe, the ultrasound imaging system, and the method of using the ultrasound imaging system of the present disclosure, it is possible to propagate an ultrasound wave between an observed region inside a subject and an ultrasound transducer in a good manner.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A guide tube comprising:
a tube that includes
a first opening arranged at a distal end;
a first lumen configured to communicate with the first opening;
a second opening that is arranged on an outer circumference on a proximal end side relative to the first opening; and
a second lumen configured to communicate with the second opening; and
a cover that is arranged on a distal end side relative to the first opening and that is arranged in a first direction when viewed from the first opening, the first direction being a direction that goes from the first lumen to the second lumen in a cross section perpendicular to a central axis of the tube.

2. The guide tube according to claim 1, wherein the cover is extended along an entire circumference of the first opening in a circumferential direction around a central axis of the first opening.

3. The guide tube according to claim 2, wherein the cover has a same outer shape as the tube in the cross section perpendicular to the central axis of the tube.

4. The guide tube according to claim 2, wherein the cover is configured with a solid material, and has an insertion hole that extends from a proximal end to a distal end, the insertion hole being configured to communicate with the first opening.

5. The guide tube according to claim 2, wherein the cover is configured with a hollow member that has a space communicating with the first opening.

6. The guide tube according to claim 5, wherein the space is filled with an ultrasound medium that propagates an ultrasound wave.

7. The guide tube according to claim 6, wherein the cover includes a third opening that allows communication between inside and outside of the space and allows injection of the ultrasound medium.

8. The guide tube according to claim 7, wherein the cover includes
a cover main body that has the third opening; and
a cap that is detachably attached to the cover main body, the cap being configured to close the third opening.

9. The guide tube according to claim 2, wherein the cover has a shape that is tapered toward a distal end.

10. The guide tube according to claim 1, wherein the second lumen includes
a lumen main body that extends along the central axis of the tube; and
an inclined portion configured to communicate with the lumen main body, the inclined portion crossing the central axis of the tube and being connected to the second opening.

11. An ultrasound probe that is assembled with a guide tube including
a tube that includes
a first opening arranged at a distal end;
a first lumen configured to communicate with the first opening;
a second opening that is arranged on an outer circumference on a proximal end side relative to the first opening; and
a second lumen configured to communicate with the second opening; and
a cover that is arranged on a distal end side relative to the first opening and that is arranged in a first direction when viewed from the first opening, the first direction being a direction that goes from the first lumen to the second lumen in a cross section perpendicular to a central axis of the tube, the ultrasound probe comprising:
a probe main body that includes an ultrasound transducer transmitting and receiving an ultrasound wave at a distal end portion of the probe main body, and that is to be inserted into the first lumen; and
a positioning mechanism that allows the distal end portion protruding from the first opening to determine a position of the probe main body with respect to the cover in the central axis direction of the tube.

12. An ultrasound imaging system comprising:
an ultrasound probe that includes an ultrasound transducer transmitting and receiving an ultrasound wave at a distal end portion of the ultrasound probe;
a treatment tool configured to perform treatment on living tissue; and
a guide tube that includes
a tube that includes
a first opening arranged at a distal end;
a first lumen in which the ultrasound probe is inserted, the first lumen being configured to communicate with the first opening;
a second opening that is arranged on an outer circumference on a proximal end side relative to the first opening; and
a second lumen in which the treatment tool is inserted, the second lumen being configured to communicate with the second opening; and
a cover that is arranged on a distal end side relative to the first opening and that is arranged in a first direction when viewed from the first opening, the first direction being a direction that goes from the first lumen to the second lumen in a cross section perpendicular to a central axis of the tube.

13. A method of using an ultrasound imaging system, the method comprising:
injecting an ultrasound medium that propagates an ultrasound wave to an inside of the cover of the guide tube according to claim 1;
inserting the ultrasound probe into the first lumen;
causing the distal end portion to protrude from the first opening;
determining a position of the ultrasound probe in a central axis direction of the tube; and
inserting the treatment tool into the second lumen.
